(19) United States
(12) Patent Application Publication
(10) Pub. No.: US 2011/0092766 A1
(43) Pub. Date: Apr. 21, 2011

(54) TAPERED LUMENS FOR MULTI-LUMEN SLEEVES USED IN ENDOSCOPIC PROCEDURES

(75) Inventors: Leonid Monassevitch, Hadera (IL); Boaz Shenhav, Tel Aviv (IL); Kobby Greenberg, Even Yehuda (IL); Dror Rosner, Holon (IL); Amol Bapaye, Pune (IN)

(73) Assignee: NITI SURGICAL SOLUTIONS LTD., Netanya (IL)

(21) Appl. No.: 12/524,044

(22) PCT Filed: Jan. 2, 2008
(86) PCT No.: PCT/IL08/00012

§ 371 Date: Jan. 6, 2011

Related U.S. Application Data
(60) Provisional application No. 60/897,247, filed on Jan. 25, 2007.

(51) Int. Cl. A61B 1/00

(52) U.S. Cl. 600/104

(57) ABSTRACT

A multi-lumen sleeve for use with an endoscope shaft and at least one working surgical instrument. The shaft and surgical instruments have an end portion. The sleeve comprises a primary lumen having a free end and one or more secondary lumens which are joined to the primary lumen. Some or all of the lumens have free ends for connection to the end portion of a selected one of the shaft and the one or more working surgical instrument. Each of the free ends of the primary and secondary lumens is adapted to fit about and support the end portion of the shaft or the one or more working surgical instrument. This facilitates concerted motion and coupled articulation between the shaft and the one or more working surgical instrument. Systems employing this multi-lumen sleeve are described.

### CROSS REFERENCE TO RELATED APPLICATION

The present application claims priority rights from U.S. Provisional Application 60/897,247, filed Jan. 25, 2007.

### FIELD OF THE INVENTION

The present invention relates to a multi-lumen sleeve having lumens with tapered ends for use in endoscopic surgical procedures.

### BACKGROUND OF THE INVENTION

Endoscopic procedures are used through much of contemporary surgery for a variety of diagnostic and therapeutic procedures. Gastrointestinal (GI) tract polyps, for example, are resected using endoscopic techniques and then biopsied. Lesions are cauterized using endoscopes.

Since there are many different uses for endoscopes, their design is varied, depending on their intended purpose. Among others, there are upper endoscopes for examination of the esophagus, stomach and duodenum, urethroscopes for examining the urethra and bladder, colonoscopes for examining the colon, laparoscopes for examining the peritoneal cavity, and sigmoidoscopes for examining the rectum and sigmoid colon.

Often, endoscopic procedures require the use of multiple working instruments. Because these multiple instruments must work in cooperation, their maneuverability and cooperation at the endoscope tip is critical to the success of the surgical procedure.

In a procedure and system discussed in “Endoscopic Full Thickness Resections Using Surgical Compression Clips”, U.S. patent application Ser. No. 11/647,912, filed Dec. 29, 2006, by the present inventors, there is described the use of a plurality of working instruments with an endoscope and this document is incorporated by reference herein. The combination of working instruments and endoscope is employed in concert using a multi-lumen sleeve.

While such multi-lumen sleeves are known, they suffer from a drawback. Working instruments are inserted into the secondary lumens and the endoscope shaft into the primary lumen of the multi-lumen sleeves. Often the working instruments are advanced into place only after the endoscope has reached its desired position. However, in those instances when advancing the endoscope and the working instruments together to the site of a lesion, the endoscope shaft and the working instruments do not necessarily proceed in tandem, the working instruments remaining somewhat behind the insertion shaft. Additionally, when the shaft is articulated the working instruments do not move in a coupled fashion with the endoscope shaft. This increases the difficulty in employing such systems in surgical procedures.

Therefore, there remains a need for a method, a system and/or elements of a system which would facilitate coupled movement and articulation between the endoscope shaft and its associated working instruments during surgical procedures.

### DEFINITIONS

“Proximal” relates to the side of the endoscope or devices closest to the user, while “distal” refers to the side of the endoscope or devices furthest from the user. Similarly, “proximal” refers to the side of the multi-lumen sleeve encasing the endoscope or of the working instruments associated with the endoscope or endoscopic system closest to the user and “distal” refers to the side furthest from the user.

“Sleeve” and “sheath” will herein be used interchangeably without intending to distinguish between them, except where specifically indicated.

“Polyp” as used in the specification and claims below is not intended to restrict the system, subsystems, elements and method discussed herein to polyps alone. Other types of suspect lesions may also be treated using the system, subsystems, elements and method discussed herein.

“Lesion” may be used in place of the word “polyp” “perforation”, hemorrhoids, tissue adjacent to a resected site, or openings within tissue generated by any surgical procedure or occurring naturally, without any intent at differentiating between these different types of lesions, except where specifically indicated.

“Gastrointestinal tract” or its equivalents may be used in the specification and claims without the intent of being limiting. Other organ systems, and lesions found therein, are also contemplated as being treatable with the system, subsystems, elements and methods discussed in the present specification.

“Working conduit”, when used in the specification and claims, may refer to a working channel of the endoscope or a secondary lumen of the sleeve whose primary lumen encases an endoscope's insertion shaft.

### SUMMARY OF THE INVENTION

It is an object of the present invention to provide a multi-lumen sleeve that allows for coupled usage of one or more working instruments with an endoscope shaft.

It is a further object of the present invention to provide a system employing a multi-lumen sleeve that allows for coupled motion and articulation between one or more working surgical instruments of the system and an endoscope shaft.

It is an object of the present invention to provide a coupling interface element for working instruments that allow for coupling motion between an endoscope and auxiliary working surgical instruments.

The endoscopic system, its sub-systems and elements, and the method described herein may find use in surgical procedures involving lesions arising in, for example, but without intending to be limiting, the bowel, rectum, appendix, gallbladder, uterus, stomach, esophagus, lungs, bladder, vagina, etc.

In one aspect of the present invention there is provided a multi-lumen sleeve for use with an endoscope shaft and one or more working surgical instruments. Each shaft and surgical instrument has an end portion. The sleeve includes a primary lumen to which is joined one or more secondary lumens. Each lumen has a free end for connection to the end portion of a selected one of the shaft and the one or more working surgical instruments. Each of the free ends of the primary and secondary lumens is adapted to fit about and support the end portion of the selected one of the shaft and the one or more working surgical instruments. This facilitates concerted translational and rotational motion and coupled articulation between the shaft and the one or more working surgical instrument.

In an embodiment of the first aspect of the present invention, one or more of the free ends of the lumens is adapted to fit about and support a coupling interface element positioned therein. The element is disengageably joined to the end portion of one of the one or more working surgical instruments.

In a second aspect of the present invention, there is provided a system for performing endoscopic surgical procedures. The system comprises an endoscope having an insertion shaft with an end portion, one or more working surgical instruments each having an end portion, and a multi-lumen sleeve. The sleeve comprises a primary lumen to which one or more secondary lumens are joined. Each of the lumens has a free end for connection to the end portion of a selected one of the shaft and the one or more working surgical instruments. Each of the free ends of the primary and secondary lumens is adapted to fit about and support the end portion of the selected one of the shaft and the one or more working surgical instruments. This facilitates concerted translational and rotational motion and coupled articulation between the shaft and the one or more working surgical instrument.

In an embodiment of the second aspect of the present invention the one or more of the free ends of the lumens is adapted to fit about and support a coupling interface element positioned therein, The element is disengageably joined to the end portion of one of the one or more working surgical instruments. In instances of this embodiment, one of the one or more working surgical instruments is extendable from the free end of one of the one or more secondary lumens after disengaging from the coupling interface element, the interface element remaining supported at the free end of the secondary lumen.

In yet a third aspect of the present invention, there is provided a system for performing endoscopic surgical procedures. The system comprises an endoscope having an insertion shaft with an end portion, a multilumen sleeve and a plurality of working surgical instruments, each of the instruments having an end portion. The multi-lumen sleeve comprises a primary lumen having a free end and one or more secondary lumens, joined to the primary lumen, and each having a free end. The plurality of working surgical instruments comprise: a compression clip for compressing tissue, the clip having an open position and a closed position, and configured to receive tissue therethrough when in its open position, and operative to apply a compression force to the tissue when closed thereabout; a clip applier in mechanical communication with the clip for advancing the clip through one of the one or more secondary lumens; and a grasper assembly selectably extendable through a selected one of the one or more secondary lumens and a working channel of the endoscope for grasping and pulling tissue through the clip when the clip is in its open position. Each of the free ends of the primary and secondary lumens is adapted to fit about and support the end portion of a selected one of the shaft and one of the plurality of working surgical instruments, thereby to facilitate concerted translational and rotational motion and coupled articulation between the shaft and one of the working surgical instruments.

In an embodiment of the third aspect of the present invention, one or more of the free ends of the lumens is adapted to fit about and support a coupling interface element positioned therein. The element is disengageably joined to the end portion of one of the one or more working surgical instruments. In instances of this last embodiment, one of the one or more working surgical instruments is extendable from the free end of one of the one or more secondary lumens after disengaging from the coupling interface element, the interface element remaining supported at the free end of the secondary lumen.

In another embodiment of the third aspect of the present invention, the system further includes a severing element for resecting tissue. The severing element is selectably extendable through a selected one of the one or more secondary lumens and a working channel of the endoscope so as to be brought into a position of operational proximity to tissue extending through the compression clip.

In yet another aspect of the present invention, there is provided a system for performing endoscopic surgical procedures for use with a multi-lumen sleeve, the sleeve having a primary lumen having a free end, and one or more secondary lumens, joined to the primary lumen, each secondary lumen having a free end. The system comprises an endoscope having an insertion shaft insertable into the primary lumen, the shaft having an end portion, and a plurality of working surgical instruments each having an end portion. The plurality of instruments comprises a clip having an open position and a closed position and configured to receive tissue therethrough when in its open position, and operative to apply a compression force to the tissue when the clip is closed thereabout; a clip applier for advancing the clip through a secondary lumen of the sleeve and for positioning the clip near the tissue to be compressed; and a grasper assembly selectably extendable through a selected one of the one or more secondary lumens and a working channel of the endoscope for engaging and pulling the tissue through the clip when the clip is in its open position. Each of the free ends of the primary and secondary lumens is adapted to fit about and support the end portion of the selected one of the shaft, the clip applier and the grasper assembly, thereby to facilitate concerted translational and rotational motion and coupled articulation between the shaft, the clip applier, and the grasper assembly.

### BRIEF DESCRIPTION OF THE DRAWINGS

The present invention will be more fully understood and its features and advantages will become apparent to those skilled in the art by reference to the ensuing description, taken in conjunction with the accompanying drawings, in which:

FIG. 1 shows a multi-lumen sleeve used with an endoscope;

FIGS. 2-5 show various configurations of multi-lumen sleeves with distended secondary lumens;

FIGS. 6-9 show the several configurations of the multi-lumen sleeves in FIGS. 2-5 with their secondary lumens collapsed;

FIG. 10 shows a schematic isometric view of a distended non-tapered multi-lumen sleeve;

FIG. 11 shows a schematic isometric view of a distended multi-lumen sleeve with tapered distal ends;

FIGS. 12A and 12B show schematic views illustrating the formation of the tapered distal ends of a multi-lumen sleeve;

FIG. 13 shows a top side view of a surgical clip attached to an applier being positioned proximate to a lesion;

FIG. 14 shows a top side view of a surgical clip and applier positioned proximate to the lesion and a tissue grasper assembly being positioned proximate to the lesion after advancing through a secondary lumen of a multi-lumen sleeve;

FIG. 15 shows a top side view of a vacuum cup of the tissue grasper assembly pulling the lesion through the clip shown in FIG. 14 after the clip has been opened;

FIG. 16 shows a top side view of the surgical clip closed around the lesion, the lesion being pulled by the vacuum cup of the tissue grasper assembly; and

FIGS. 17-20 show various stages of extending the working instruments from a multi-lumen sleeve with tapered ends and the resulting coupled articulation.

Similar elements in the Figures are numbered with similar reference numerals.

### DETAILED DESCRIPTION OF THE PREFERRED EMBODIMENTS

The sleeve, also sometimes denoted herein as the sheath, of the present invention is a multi-lumen sleeve with an endoscopic insertion shaft positioned in the sleeve's primary lumen and one or more working instruments, such as, but not limited to a clip and its applier, a severing instrument and a grasper assembly, positioned in one or more secondary lumens of the sleeve. At the distal end of the sleeve the lumens of the sleeve are tapered or otherwise adapted to connect to, fit about and support the distal ends of the endoscope shaft or working instruments. When the distal ends of the lumens are tapered they wedge the working instruments into the lumens of the sleeve. They advance in concert with the endoscope shaft and importantly, also articulate in tandem with the endoscope shaft. In cases where the tapering is insufficient or inefficient, there may be a coupling interface element around a working instrument which allows the working instrument to be wedged in the secondary lumen as required for coupled motion and articulation.

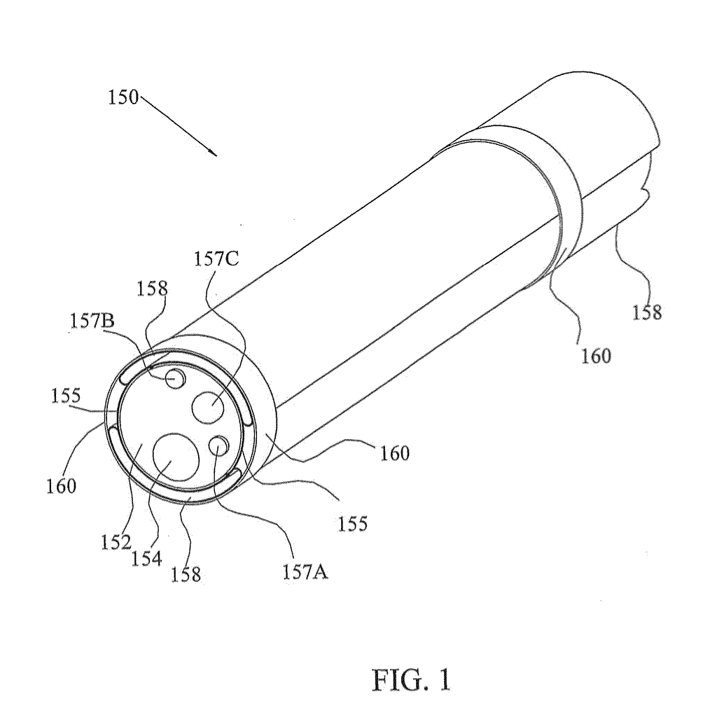

Multi-lumen sleeves are shown in FIGS. 1-9, to which reference is now made.

FIG. 1 shows an isometric view of the distal end 152 of the insertion shaft of an endoscope positioned in a primary lumen 155 of a multi-lumen sleeve 150. The sleeve 150 has a primary lumen 155 and at least one secondary lumen 158. Both the distal end and the proximal ends of the sleeve may be open.

The distal end 152 of the insertion shaft of the endoscope includes a working channel 154, and at least one auxiliary element 157, such as optics, illumination, irrigation etc. In FIG. 1, three such auxiliary elements 157 are present, but more or fewer auxiliary elements may be present in other embodiments.

When inserting the sleeve-encased endoscope into a patient, the secondary lumens 158 typically but without being limiting, are collapsed. Keeping the secondary lumens collapsed allows for a smaller profile as the endoscope is inserted into a body cavity, wending its way toward a lesion. One method of keeping the secondary lumens collapsed and substantially adjacent to the primary lumen 155 is by using bands 160, typically, but without intending to be limiting, bands made of silicone.

The multi-lumen sleeve 150 can be made of any of many different types of flexible plastics. Without intending to limit the choice of flexible plastics or elastomers, these may include polyethylene, polyurethane, polyvinyl chloride and almost any other medical grade plastic.

Secondary lumens 158 may be formed using any of several known methods for working sheet plastics; most typically the secondary lumens 158 are formed integrally with the primary lumen. The secondary lumens can be kept collapsed by directly extruding the multi-lumen sleeve with the secondary lumens in their collapsed positions. Secondary lumen(s) may also be attached to a primary lumen using one of many techniques known to those skilled in the art such as by using a suitable medical grade glue or solvent, by employing soldering, by heat treatment, or by using high frequency welding.

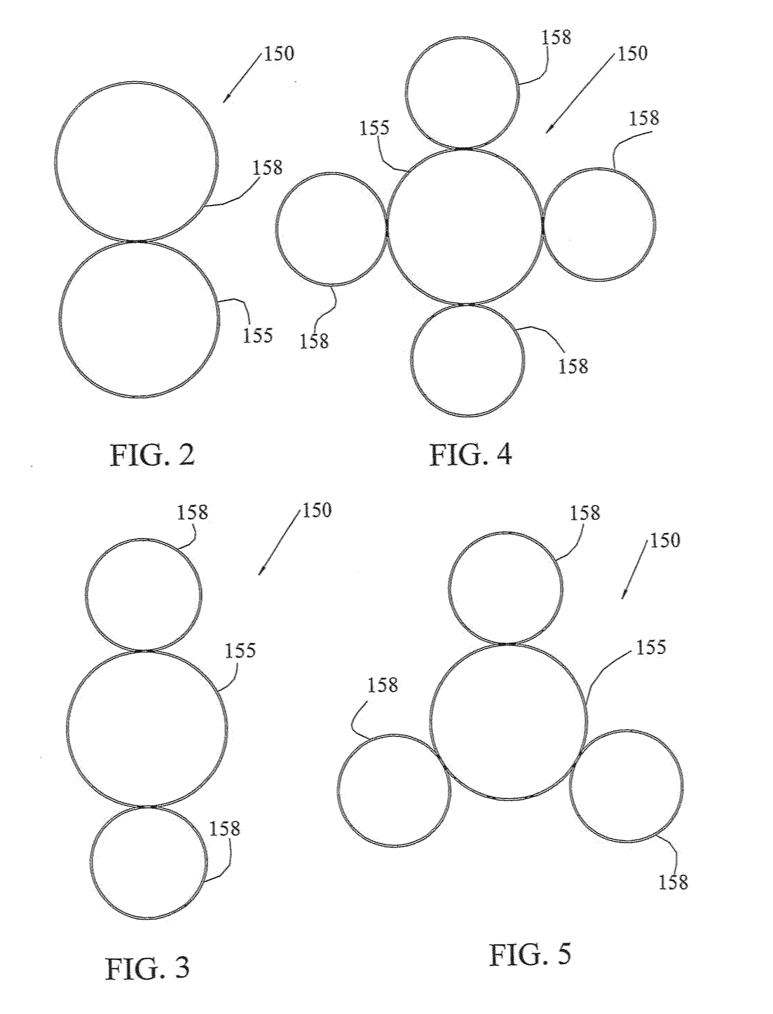

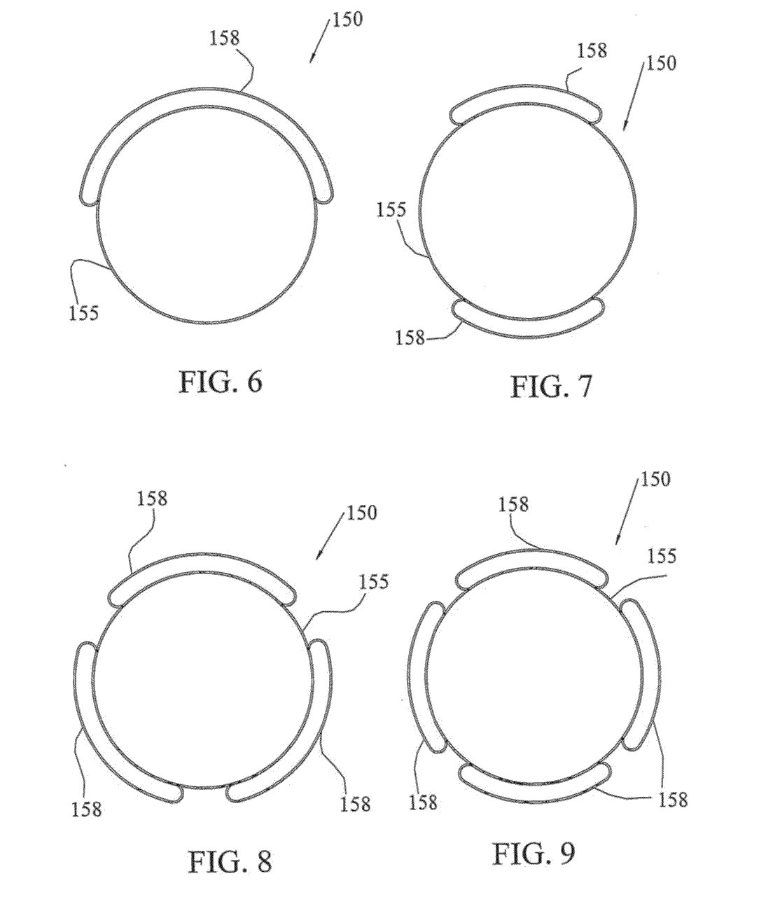

High frequency (HF) plastic welding may be used to weld multiple single secondary lumens to the primary lumen producing sleeve shapes shown in FIGS. 2-5. The sleeve can also be welded from a single plastic sheet, after first doubling back the sleeve one or more times so that portions of the sleeve are positioned to be adjacent to each other. HF welding may be used to keep the secondary lumens in their collapsed state as in FIG. 6-9, by using a “gentle” weld also known as floating welding. As instruments are passed through the secondary lumen, the floating weld breaks and the lumen distends.

FIGS. 2-9 show various configurations of multi-lumen sleeves 150 usable with the present invention. As noted above, the secondary lumens 158 may be integrally formed with the primary lumen 155 by extrusion or by any of a number of plastic sheet processing techniques, such as by hot welding or high frequency (HF) welding or solvent gluing. The attachment should be effected so that it allows expansion of the secondary lumen(s) 158 when surgical instruments pass through them. The number of secondary lumens 158 is different in each of the configurations shown. The secondary lumens 158 are shown in their distended state in FIGS. 2-5, as is the case when surgical working tools are positioned inside them. FIGS. 6-9 show various configurations of integrally formed multi-lumen sleeves 150 with the number of secondary lumens 158 different in each configuration. In FIGS. 6-9 the secondary lumens 158 are collapsed.

While in the embodiments described above the primary lumen is continuous, in other embodiments it need not be. In these other embodiments, the primary lumen may include holes, be net-like, etc.

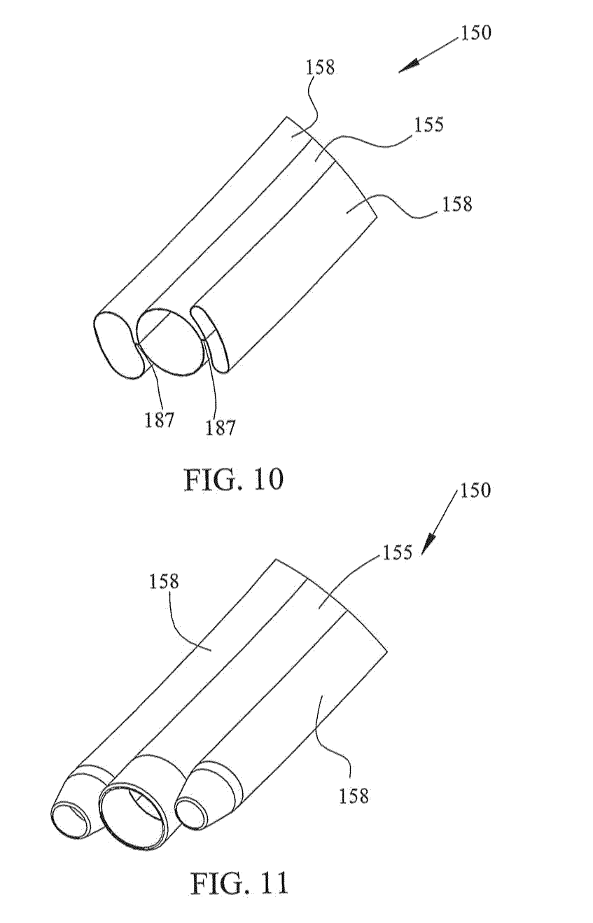

Reference is now made to FIGS. 10-11 where two isometric views of multi-lumen sleeves are shown. FIG. 10 shows a sleeve with untapered distal ends. FIG. 11 shows an isometric view of a multi-lumen sleeve constructed according to the present invention with primary lumen 155 and secondary lumens 158 all being tapered at each of their distal ends. In some embodiments, not all of the lumens have to be tapered. Using tapered lumens allows for the insertion into the body of the endoscope shaft in concert with any needed working instrument(s). Typically, it does not require the use of bands as described in the embodiment shown in and discussed above in conjunction with the sleeve shown in FIG. 1.

The present invention as presented in FIG. 11 teaches that in order to ensure control over the maneuverability of an auxiliary working instrument, such as a clip and a clip applier (not shown), coupling is required between the endoscope and the auxiliary working instrument. The diameter of the distal end of secondary lumens 158 may be reduced to fit snugly over the outer diameter of the instruments being delivered through these lumens. When an auxiliary working instrument is inserted into a secondary lumen 158 and reaches its distal end, the outer diameter of the instrument fits tightly into the distal end of the secondary lumen 158. This ensures a coordinated motion of the endoscope and the auxiliary working instruments. The motion and articulation of the auxiliary working instrument will be coupled with the motion and articulation of the tip of the endoscope as a result of the snug fits of both in their respective sleeve lumens.

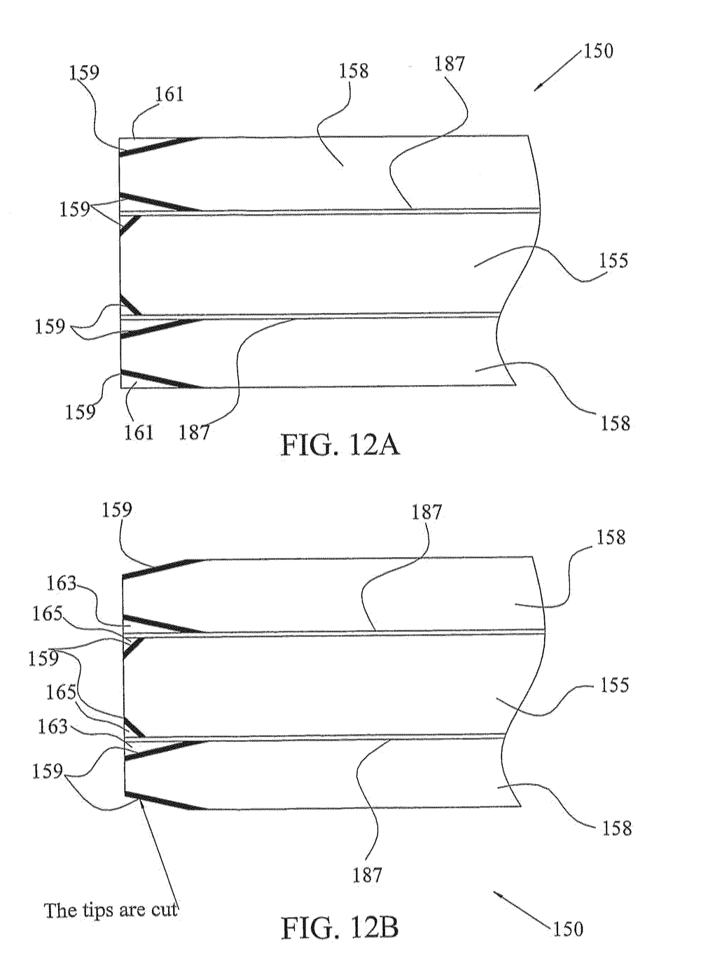

Reference is now made to FIGS. 12A and 12B which illustrate a typical, but non-limiting, method for constructing the tapered distal lumen ends of the present invention. In FIG. 12A, a planar top view of a plastic multi-lumen sleeve is shown. The sleeve has been constructed by one of the methods discussed above. Secondary lumens 158 are joined to primary lumen 155 along juncture lines 187. Additionally, there are heat generated diagonal welds shown as bolded diagonal lines 159 that constrict or taper the distal ends of primary 155 and secondary 158 lumens. In FIG. 12B, the excess plastic of triangular areas 161 have been cut to reduce the profile of sleeve 150.

In some embodiments, not all of bolded lines 159 are heat welded. If some lumens do not require tapered, constricted ends, lines 159 are not heat welded. In other embodiments, only one line 159 per lumen may be heat welded. This controls the position of the coupling element discussed herein below and thus the position of the working instruments relative to the endoscope and its working channel. In yet other embodiments, the excess plastic in triangular areas 163 or 165 or both may also be cut off.

The tapered constricted distal end of a secondary lumen can be formed so as to fit the size, that is the outer diameter, of the working instrument expected to be inserted into that lumen. Similarly, the reduced diameter of a primary lumen can be formed so as to fit the size, the outer diameter, of the endoscope to be inserted into that lumen. Similarly, the reduced diameter of a secondary lumen can be formed so as to fit the size of a coupling element (as discussed below) disengagably joined to a working instrument. Similarly, the reduced diameter of a secondary lumen can be formed so as to fit the size of a covering or casing associated with a working instrument.

While heat welding as shown by diagonal bold lines 159 in FIGS. 12A-12B can be used to constrict or taper the lumens of a multi-lumen sleeve, this method is not intended to be limiting. The reduction of the diameter of the distal end of the primary 155 and/or the secondary 158 lumens of sleeve 150 can be achieved in a variety of other ways. These include, but are not limited to, heat welding, warm soldering—symmetrical or unsymmetrical—of the lumens, welding of cone-shaped tips to the distal end of the sleeve using material similar to the sleeve itself, special heat treatment to the distal end of the lumens, gluing, high frequency welding, etc. Additionally, a ring made of a plastic with a greater hardness or greater thickness than that of the sleeve may be welded to the tip of the sleeve. The ring should have a hole with a smaller diameter than the sleeve's tip.

In general, in endoscopic surgical procedures, the primary lumen of the sleeve should be fitted over the endoscope's insertion shaft in such a way that there is no relative axial movement between the lumen and endoscope shaft. As noted above, this can be achieved by using various techniques, including but not limited to, the use of bands. However, it may also be achieved by reducing the primary lumen's 155 diameter at its distal end by the method discussed in conjunction with FIGS. 12A-12B. In the latter technique, the sleeve's distal end diameter is reduced by the use of diagonal welds 159 so that lumen 155 fits tightly over the distal end of the endoscope. This may eliminate the need for additional connection accessories such as bands.

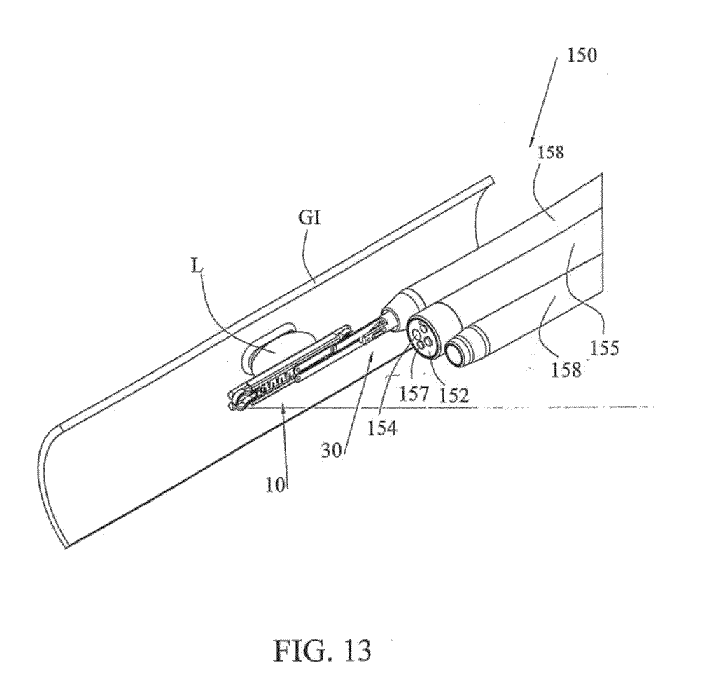

FIGS. 13-16, to which reference is now made, illustrate a typical use of the tapered multi-lumen sleeve shown in and discussed in conjunction with FIG. 11.

FIG. 13 shows an endoscope insertion shaft E (not shown being inside primary lumen 155) having endoscope distal face 152 in primary lumen 155 of tapered sleeve 150. Endoscope insertion shaft E includes a working channel 154. It also contains several auxiliary elements, here three, denoted as 157. The number of working and auxiliary channels may be more or less than three in other embodiments of shaft E. A multi-lumen plastic sleeve 150 is brought to and placed over endoscope insertion shaft E so that shaft E is encased in the primary lumen 155 of multi-lumen sleeve 150. Clip 10, attached to clip applier 30, is extended past the tapered distal end of secondary lumen 158 and brought, while still in its closed position, near lesion L.

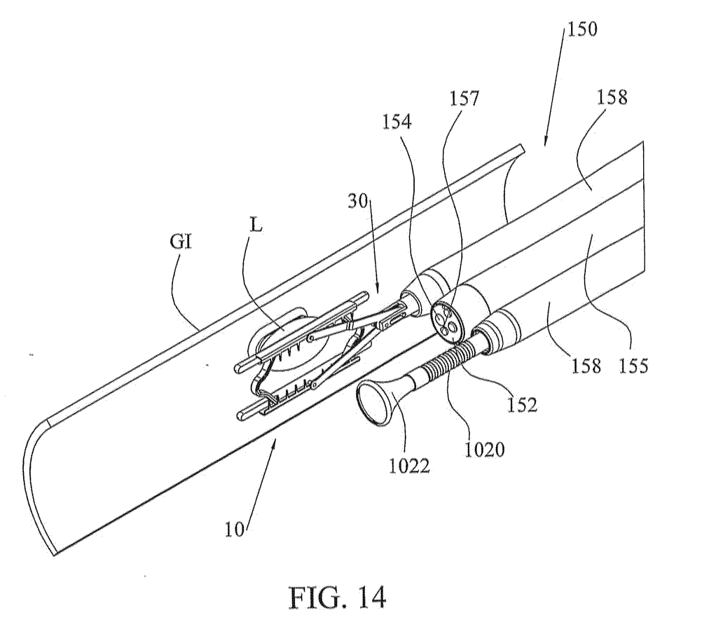

Turning to FIG. 14, a tissue grasper assembly, positioned in a second secondary lumen 158 of multi-lumen sleeve 150, is advanced through the lumen and past the distal end 152 of endoscope insertion shaft E to the region adjacent to lesion L.

Clip 10 is then opened by applier 30 in order to pull lesion L through the clip. FIG. 14 shows an isometric view of the opened clip.

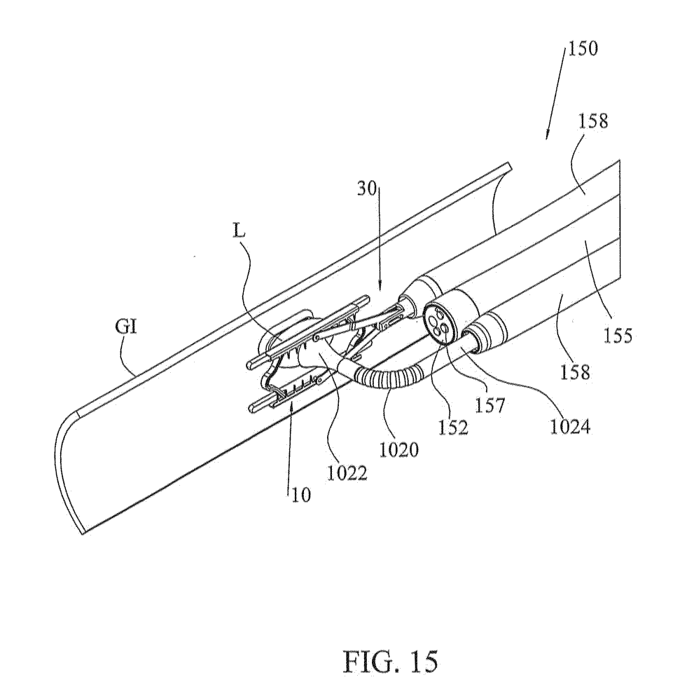

Until clip 10 is opened and positioned close to lesion L, the grasper remains within a connector tube (not shown) inside lumen 158. After clip 10 is opened, vacuum cup 1022 advances out of lumen 158 and opens in stages. Using an articulation wire to maneuver flexible cup transporter 1020, here formed having a spring construction, vacuum cup 1022 is positioned to grasp lesion L through open clip 10 (FIG. 15). In FIGS. 14-16, the articulation wire is obscured by vacuum cup 1022, cup transporter 1020, and connector tube 1024.

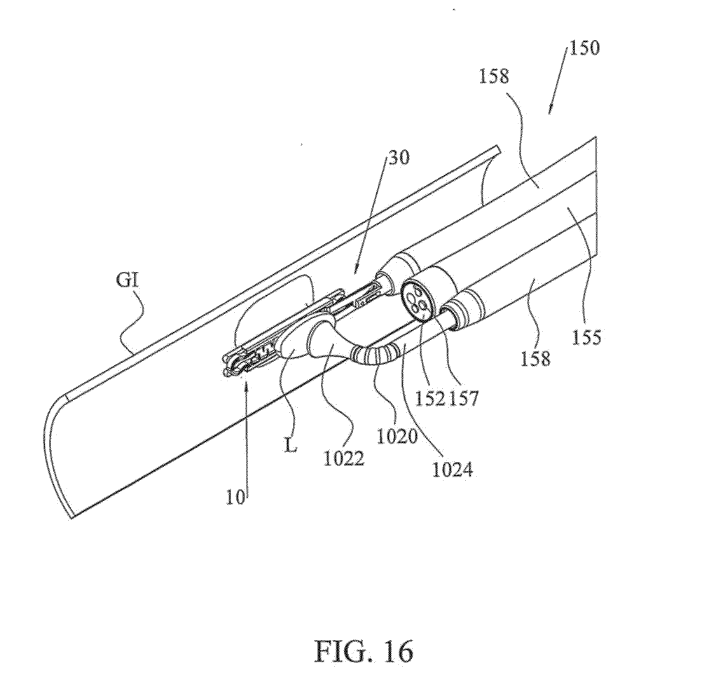

Lesion L is then pulled by vacuum cup 1022 through open clip 10, and subsequently, clip applier 30 closes clip 10 around pulled lesion L. This is shown in an isometric view in FIG. 16. Then, clip applier 30 is detached from the closed clip 10 and withdrawn via the secondary lumen 158 through which it entered (not shown).

Lesion L compressed by clip 10 may be severed by a severing device 310 (not shown) introduced through a working channel of endoscope E or through another secondary lumen of the sleeve. The actual step of severing is not shown.

After severance of lesion L, the severed polyp held by the vacuum cup 1022 of the grasper, together with the remainder of the grasper assembly, the severing device 310 and the endoscope shaft, are retracted in the direction of the proximal end of the endoscope and withdrawn from the body. Withdrawal directly from the body organ is a straight-forward step, and therefore this step is not presented in a separate Figure. Lesion L can then be biopsied or treated as needed by a physician.

The closed surgical compression clip 10 remains around that portion of the GI wall from which the lesion L was resected. Compression continues until necrosis is induced and healing of the resected site occurs. Clip 10 is naturally expelled from the body through the rectum.

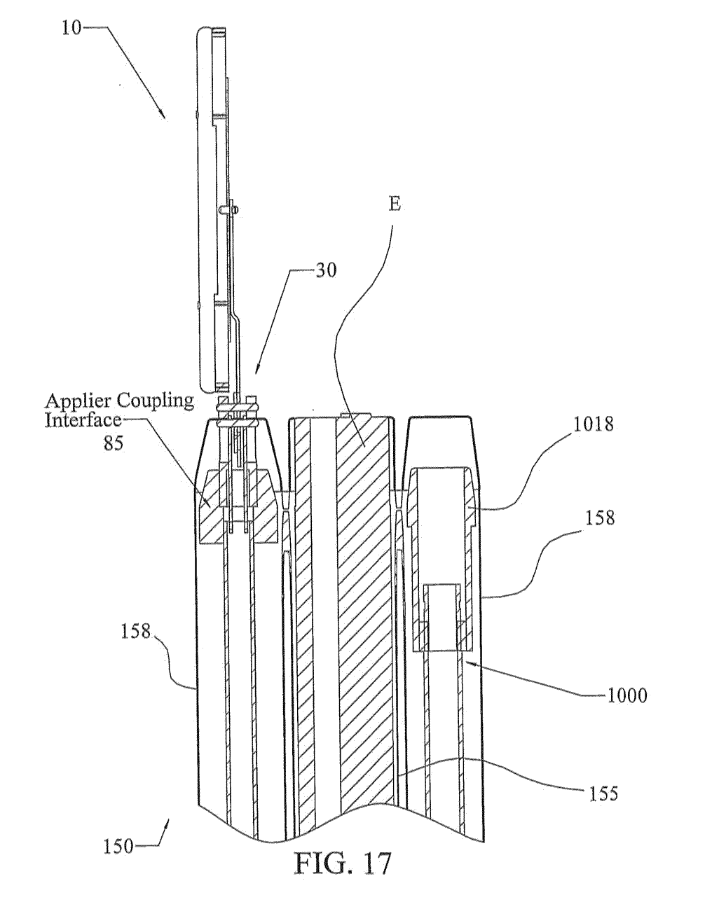

Reference is now made to FIGS. 17-20 wherein use of a multi-lumen sleeve constructed according to the present invention is shown. In FIG. 17, a vacuum cup assembly 1000 is positioned in one secondary lumen 158 of sleeve 150, an endoscope E is positioned in a primary lumen 155 of sleeve 150, and a clip 10 joined to a clip applier 30 is positioned in a second secondary lumen 158 of sleeve 150.

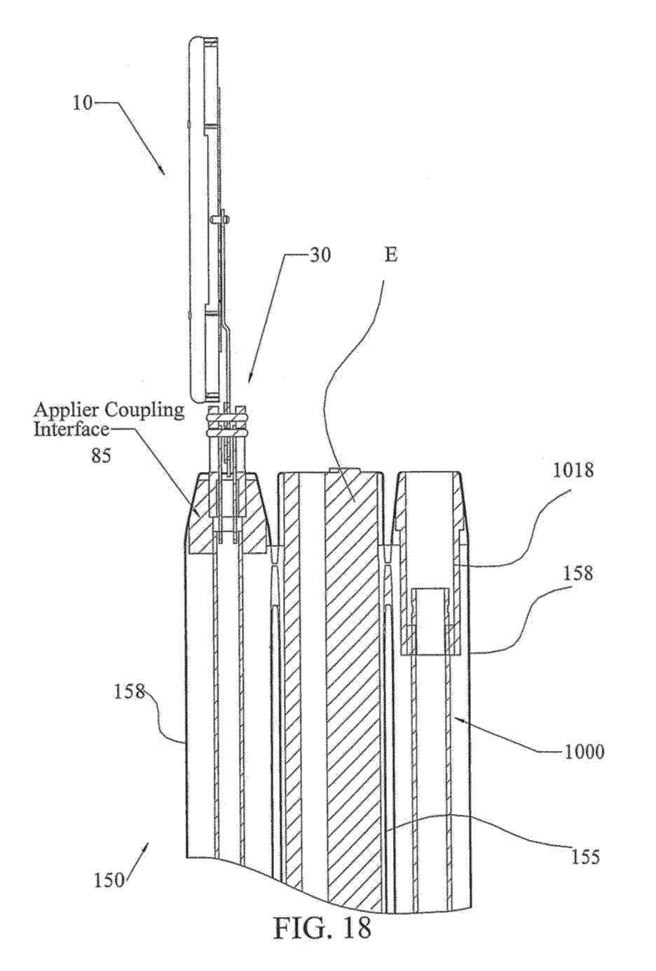

The vacuum cup cover 1018 of grasper assembly 1000 is brought to the tapered end of secondary lumen 158 (FIG. 17) and effectively wedged therein (FIG. 18). Similarly, clip 10 with attached clip applier 30 is brought to the tapered end of another secondary lumen 158 (FIG. 17) and wedged therein (FIG. 18). Joined to clip applier 30 is coupling interface element 85 which in FIG. 18 is the part of applier 30 that is actually wedged in at the tapered end of lumen 158. When in position near the site of a lesion, coupling interface element 85 allows rotation of applier 30 within element 85 to arrive at the desired orientation for operation of clip 10. Typically, there is a preferred orientation with respect to the lesion when using the instrument. Without intending to be limiting, there are various ways of controlling orientation such as a swivel mechanism or by rotating the entire instrument. In both FIGS. 17 and 18, endoscope shaft E is wedged at the tapered distal end of primary lumen 155.

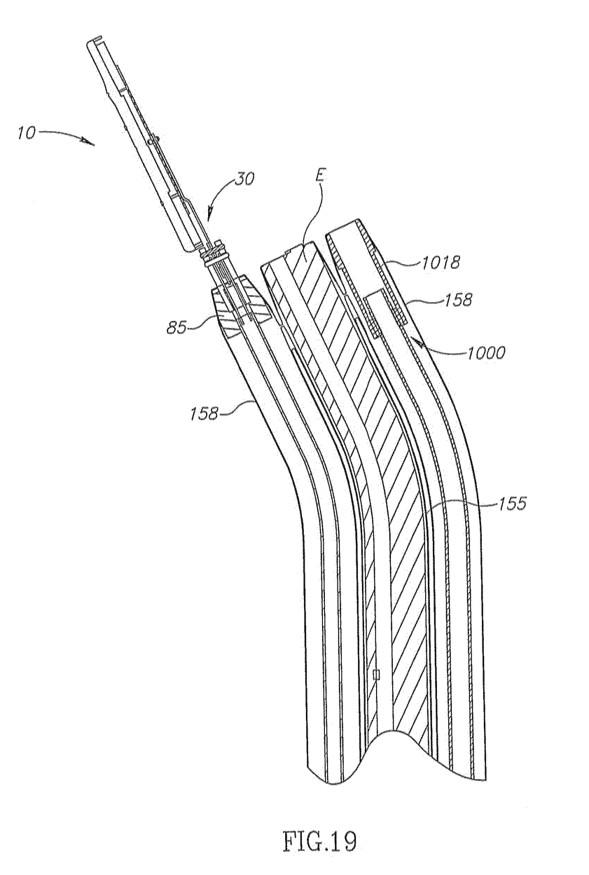

With endoscope E, vacuum cup cover 1018 of grasper assembly 1000 and coupling interface element 85 attached to clip applier 30 all wedged in their respective lumens, it is readily evident that coupled motion and articulation of the two working instruments and endoscope E as shown in FIG. 19 is achieved. Such movement and articulation is required in order to bring the instruments to their desired point of operation and their desired orientation vis-à-vis a lesion.

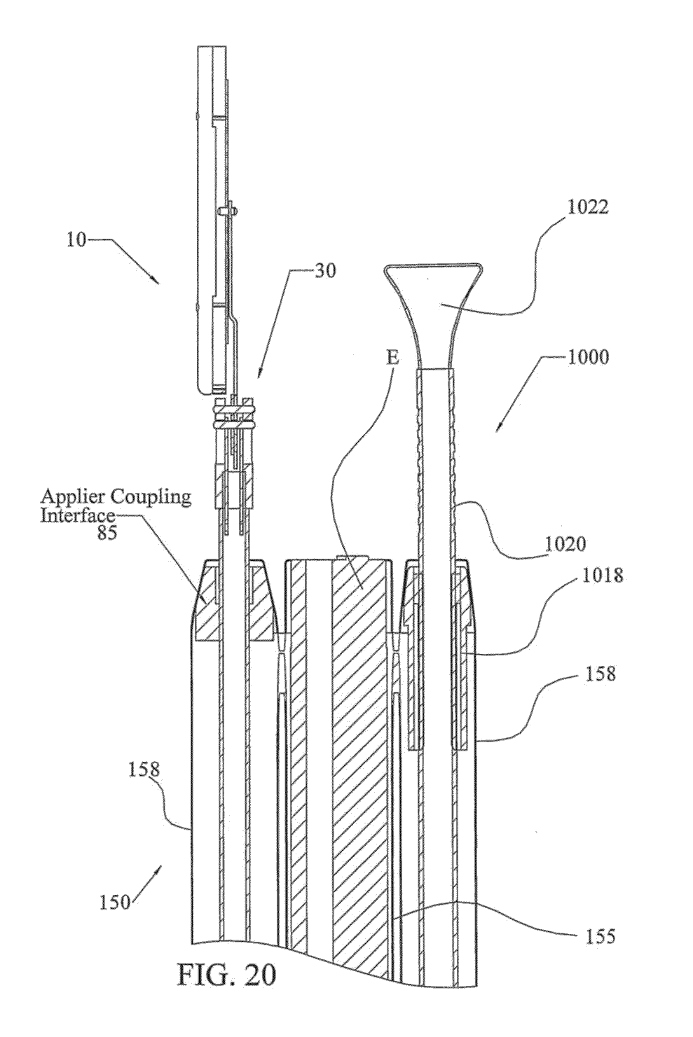

FIG. 20 shows the extension of vacuum cup 1022 out from vacuum cup cover 1018, the latter still wedged at the tapered end of secondary lumen 158. The Figure also shows that clip applier 30 has disengaged from coupling interface element 85, the latter still wedged at the tapered end of its secondary lumen 158. At this stage coupling of the instruments between themselves and for the endoscope is “broken”.

When the clip applier is withdrawn it may be pulled back and reengaged with coupling interface element 85 and the applier 30, and element 85 together may be withdrawn from the lumen by pulling both in the lumens proximal direction.

In FIGS. 17-20 there is presented a coupling interface element 85 to ensure coupling of one instrument with another instrument and/or with the endoscope shaft E. In other cases, as in the case of grasper assembly 1000, a part of the working instrument itself can serve to couple the instrument with another instrument and/or with the endoscope shaft.

FIGS. 17-20 illustrate an embodiment where there is a single coupling interface element positioned in a secondary lumen of the multi-lumen sleeve. In another embodiment, the single coupling element may be positioned in the primary lumen of the sleeve. In yet another embodiment, there may be more than one coupling element, each element positioned in a different secondary lumen. In a further embodiment, there may be more than one coupling element, one element positioned in the primary lumen and each of the remaining elements positioned in a different secondary lumen. Finally, in still another embodiment, each lumen, that is the primary lumen and each of the secondary lumens, may contain a coupling interface element within it.

It should be evident to one skilled in the art that the present invention can be used in surgical procedures of many different organs in many different organ systems, with little or no modification. Such organs include, but are not limited to, the bowel and rectum and other organs of the gastrointestinal (GI) tract, the urinary bladder and other organs of the urinary tract, the uterus, the liver, the esophagus, the gall bladder, and the lungs.

“Endoscope”, as used herein, contemplates the use of the present invention with all different types of invasive instruments, flexible or rigid, having scope features. These include, but are not limited to, instruments referred to as endoscopes, colonoscopes, gastroscopes, laparoscopes, and rectoscopes. Such instruments, as is readily known to those skilled in the art, are subsumed within the term endoscope. The present invention, while discussed in terms of endoscopes can readily be adapted for use with each of these instruments with little or no modification. It should also be noted that the use of the term “endoscopic” is to be construed as referring to the many different types of invasive scopes subsumed under the term endoscopes. As known by those skilled in the art the term “invasive” denotes a medical procedure requiring insertion of an instrument or device into the body through the skin or a body orifice for diagnosis or treatment.

It should be readily apparent to one skilled in the art that the device and method of the present invention can be used in surgical procedures on animals, particularly mammals, as well as on humans.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention.

It will be appreciated by persons skilled in the art that the present invention is not limited by the drawings and description hereinabove presented. Rather, the invention is defined solely by the claims that follow.

1. A multi-lumen sleeve for use with an endoscope shaft and at least one working surgical instrument, each shaft and surgical instrument having an end portion, wherein said sleeve comprises:
a primary lumen having a free end for connection to the end portion of a selected one of the shaft and the at least one working surgical instrument; and
at least one secondary lumen joined to said primary lumen, and having a free end for connection to the end portion of a selected one of the shaft and the at least one working surgical instrument,
wherein each of said free end of said primary and secondary lumens is adapted to fit about and support the end portion of the selected one of the shaft and the at least one working surgical instrument, thereby to facilitate concerted translational and rotational motion and coupled articulation between the shaft and the at least one working surgical instrument.

2. A multi-lumen sleeve according to claim 1 wherein at least one of said free ends of said lumens is adapted to fit about and support a coupling interface element positioned therein, said element disengageably joined to said end portion of one of said at least one working surgical instrument.

3. A system for performing endoscopic surgical procedures, which comprises:
an endoscope having an insertion shaft with an end portion;
at least one working surgical instrument having an end portion; and
a multi-lumen sleeve comprising:
a primary lumen having a free end for connection to the end portion of a selected one of the shaft and the at least one working surgical instrument; and
at least one secondary lumen joined to said primary lumen, and having a free end for connection to the end portion of a selected one of the shaft and the at least one working surgical instrument,
wherein each of said free ends of said primary and secondary lumens is adapted to fit about and support said end portion of the selected one of said shaft and said at least one working surgical instrument, thereby to facilitate concerted translational and rotational motion and coupled articulation between said shaft and said at least one working surgical instrument.

4. A system according to claim 3 wherein at least one of said free ends of said lumens is adapted to fit about and support a coupling interface element positioned therein, said element disengageably joined to said end portion of one of said at least one working surgical instrument.

5. A system according to claim 4 wherein said one of said at least one working surgical instrument is extendable from said free end of one of said at least one secondary lumens after disengaging from said coupling interface element, said interface element remaining supported at said free end of said secondary lumen.

6. A system for performing endoscopic surgical procedures, which comprises:
an endoscope having an insertion shaft with an end portion;
a multi-lumen sleeve comprising:
a primary lumen having a free end; and
at least one secondary lumen, joined to said primary lumen, and each of said at least one secondary lumen having a free end; and
a plurality of working surgical instruments, each having an end portion, comprising:
a compression clip for compressing tissue, said clip having an open position and a closed position, and configured to receive tissue therethrough when in its open position, and operative to apply a compression force to the tissue when closed thereabout;
a clip applier in mechanical communication with said clip and for advancing said clip through one of said at least one secondary lumens; and
a grasper assembly selectably extendable through a selected one of said at least one secondary lumens and a working channel of the endoscope for grasping and pulling tissue through said clip when said clip is in its open position, and
wherein each of said free ends of said primary and secondary lumens is adapted to fit about and support said end portion of a selected one of said shaft and one of said plurality of working surgical instruments, thereby to facilitate concerted translational and rotational motion and coupled articulation between said shaft and said working surgical instruments.

7. A system according to claim 6 wherein at least one of said free ends of said lumens is adapted to fit about and support a coupling interface element position therein, said element disengageably joined to said end portion of one of said at least one working surgical instrument.

8. A system according to claim 7 wherein said one of said at least one working surgical instrument is extendable from said free end of one of said at least one secondary lumens after disengaging from said coupling interface element, said interface element remaining supported at said free end of said secondary lumen.

9. A system for performing endoscopic surgical procedures according to claim 6, which further includes a severing element for resecting tissue, selectably extendable through a selected one of said at least one secondary lumens and a working channel of the endoscope so as to be brought into a position of operational proximity to tissue extending through said compression clip.

10. (canceled)
